(19) United States
(12) Patent Application Publication
(10) Pub. No.: US 2005/0165401 A1
(43) Pub. Date: Jul. 28, 2005

(54) Pelvic fixation plate

(76) Inventor: Larry Pack, Grand Blanc, MI (US)

Correspondence Address: Warn, Burgess & Hoffmann, P.C., P.O. Box 70098, Rochester Hills, MI, 48307, US

(21) Appl. No.: 10/765,237

(22) Filed: Jan. 26, 2004

(51) Int. Cl. A61B 17/56

(52) U.S. Cl. 606/69

(57) ABSTRACT

A reconstruction plate for reconstructing a load-bearing anatomical structure, such as a fractured acetabulum, and methods for using the same, are described. The plate primarily includes: (1) an attachment member having at least one aperture formed therein; (2) an optional additional attachment member extending from the attachment member, the additional attachment member having at least one aperture formed therein; and (3) a support member extending angularly from the attachment member. The support member is operable to provide support to a posterior aspect of the reconstructed acetabulum during loading of the reconstructed acetabulum, e.g., during standing, walking, running, jumping, and the like.

### FIELD OF THE INVENTION

The present invention relates generally to orthopedic internal fixation devices, and more particularly to a pelvic fixation plate suitable for reconstructing acetabular fractures, especially fractures involving the acetabular dome, and methods for using the same.

### BACKGROUND OF THE INVENTION

The use of orthopedic devices, such as reconstruction plates and the like, to repair fractures of the pelvis, including the acetabulum, is well known in the art. Examples of various orthopedic devices can be found with reference to U.S. Pat. No. 4,454,876 to Mears; U.S. Pat. No. 4,573,458 to Lower; U.S. Pat. No. 4,800,874 to David et al.; U.S. Pat. No. 4,955,886 to Pawluk; U.S. Pat. No. 4,959,065 to Arnett et al.; U.S. Pat. No. 5,336,224 to Selman; U.S. Pat. No. 5,487,741 to Maruyama et al.; U.S. Pat. No. 5,690,631 to Duncan et al.; U.S. Pat. No. 5,718,705 to Sammarco; U.S. Pat. No. 5,746,742 to Runciman et al.; U.S. Pat. No. 5,752,958 to Wellisz; U.S. Pat. No. 5,766,175 to Martinotti; U.S. Pat. No. 5,904,684 to Rooks; U.S. Pat. No. 5,984,925 to Apgar; U.S. Pat. No. 6,004,353 to Masini; U.S. Pat. No. 6,123,709 to Jones; U.S. Pat. No. 6,238,969 to Grusin et al.; U.S. Pat. No. 6,306,173 to Masini; U.S. Pat. No. 6,340,362 to Pierer et al.; U.S. Pat. No. 6,348,052 to Sammarco; U.S. Pat. No. 6,364,881 to Apgar et al.; U.S. Pat. No. 6,440,131 to Haidukewych; and U.S. Patent Application Publication 2002/0128653 to Haidukewych, the entire specifications of all of which are expressly incorporated herein by reference.

Of particular interest to orthopedic surgeons is the repair of fractures involving the acetabulum. The acetabulum is formed from three ossification centers: the ilium, ischium, and pubis. Each contributes, in part, to the acetabulum's development at the triradiate cartilage. The primary anatomical components of the acetabulum are the columns, walls, dome, and quadrilateral plate. The dome of the acetabulum is the superior aspect that carries most of the weight-bearing forces.

One function of the acetabulum is to provide a means for the transfer of weight-bearing forces from the appendicular to axial skeleton via its articulation with the femoral head. This same femoral head transfers high-energy forces to the acetabulum in the setting of trauma. The pattern of acetabular injury is determined by the position of the femoral head at the time of the traumatic event. For example, when the femoral head is adducted, the force is transmitted to the acetabular dome.

Fractures of the acetabular dome, especially T-type fractures, are difficult to treat due, in part, to the requirement that the reconstructed acetabular dome is capable of tolerating applied loads caused by normal everyday activities, such as standing, walking, running, jumping, and the like. A conventional surgical approach to pelvic fractures involving the acetabular dome is to employ an anterior reconstruction plate along the anterior aspect of the pelvis and a posterior reconstruction plate along the posterior aspect of the pelvis, both typically in proximity to the fracture site. Although this approach supplies some stability to the reconstructed pelvis, it does not provide the requisite support for the acetabulum, especially the area of the acetabular dome, in order to withstand repeated loads placed thereon. In certain circumstances, the posterior reconstruction plate may fail, potentially allowing the femoral head to penetrate through the patieni's fractured acetabulum and into the pelvis, thus necessitating even further and more complicated surgical intervention.

Therefore, there exists a need for a new and improved pelvic fixation plate that is operable to provide sufficient support to the acetabulum, especially the posterior aspect thereof, e.g., during loading of the acetabular dome area.

### SUMMARY OF THE INVENTION

In accordance with the general teachings of the present invention, a new and improved reconstruction plate is provided for reconstructing fractured load-bearing anatomical structures. More specifically, the reconstruction plate is intended to be used as a pelvic fixation plate in order to repair pelvic fractures. The reconstruction plate of the present invention is especially suitable for reconstructing acetabular fractures, including, but not limited to fractures involving the dome or roof of the acetabulum.

The reconstruction plate, in its broadest terms, generally comprises a attachment member having at least one, and more preferably, a plurality of apertures formed therein. The intended purpose of the apertures is to receive fastening members, such as bone screws, so as to enable the fixation of the reconstruction plate to one or more surfaces of the fracture site and surrounding areas. The attachment member is preferably fastened in proximity to an anterior aspect of the fracture site and surrounding areas, e.g., the anterior aspect of the acetabulum. By way of a non-limiting example, the attachment member is preferably fastened in proximity to the iliopubic eminence.

Optionally, one or more additional attachment members may be provided, wherein the additional attachment members extend away from the attachment member, e.g., substantially perpendicular to and/or co-planar with the attachment member. The additional attachment member can include at least one, and more preferably, a plurality of apertures formed therein, for receiving fastening members. The intended purpose of the additional attachment member is to provide additional fixation points so as to increase the fixation strength of the reconstruction plate, as well as to provide the orthopedic surgeon more versatile fixation options. The additional attachment member is preferably fastened in proximity to an anterior aspect of the fracture site and surrounding areas, e.g., the anterior aspect of the acetabulum. By way of a non-limiting example, the additional attachment member is preferably fastened in proximity to the iliopubic eminence.

Angularly extending from the attachment member is a support member. The exact shape or configuration of the support member may be varied provided that the structure provides a sufficient amount of support to the posterior aspect of the reconstructed anatomical structure. By way of a non-limiting example, a paddle-like shape is one preferred shape of the support member. Additionally, the support member may be positioned anywhere along the length of the attachment member, although it is preferred that the support member is located substantially at the midpoint of the attachment member. The intended purpose of the support member is to provide support (e.g., mechanical) to a posterior aspect of the fracture site when the reconstructed load-bearing anatomical structure (e.g., an acetabulum) is loaded (e.g., when standing, walking, jumping, and the like). By way of a non-limiting example, the support member is preferably positioned in proximity to the iliopectineal line.

In accordance with a first embodiment of the present invention, a reconstruction plate for reconstructing a fractured load-bearing anatomical structure is provided, comprising: (1) an attachment member including at least one area defining an aperture formed therein; and (2) a support member extending angularly from the attachment member. The support member is operable to provide support to a posterior aspect of the reconstructed anatomical structure during loading of the reconstructed anatomical structure.

In accordance with a second embodiment of the present invention, a reconstruction plate for reconstructing a fractured acetabulum is provided, comprising: (1) an attachment member including at least one area defining an aperture formed therein; and (2) a support member extending angularly from the attachment member. The support member is operable to provide support to a posterior aspect of the reconstructed acetabulum during loading of the reconstructed acetabulum.

In accordance with a third embodiment of the present invention, a reconstruction plate for reconstructing a fractured acetabulum is provided, comprising: (1) an attachment member including a plurality of areas defining apertures formed therein; (2) at least one other attachment member extending substantially perpendicularly from the attachment member, the at least one other attachment member including at least one area defining an aperture formed therein; and (3) a support member extending angularly from the attachment member. The support member is operable to provide support to a posterior aspect of the reconstructed acetabulum during loading of the reconstructed acetabulum.

In accordance with a fourth embodiment of the present invention, a method for reconstructing a fractured load-bearing anatomical structure is provided, comprising: (1) providing a reconstruction plate, comprising: (a) an attachment member including at least one area defining an aperture formed therein; and (b) a support member extending angularly from the attachment member; and (2) fastening the reconstruction plate in proximity to the anterior aspect of the fractured anatomical structure so as to at least partially reconstruct the fractured anatomical structure. The support member is operable to provide support to the posterior aspect of the reconstructed anatomical structure during loading of the reconstructed anatomical structure.

In accordance with a fifth embodiment of the present invention, a method for reconstructing a fractured acetabulum is provided, comprising: (1) providing a reconstruction plate, comprising: (a) an attachment member including at least one area defining an aperture formed therein; and (b) a support member extending angularly from the attachment member; and (2) fastening the reconstruction plate in proximity to the anterior aspect of the fractured acetabulum so as to at least partially reconstruct the fractured acetabulum. The support member is operable to provide support to the posterior aspect of the reconstructed acetabulum during loading of the reconstructed acetabulum.

A more complete appreciation of the present invention and its scope can be obtained from the following detailed description of the invention, the drawings, and the appended claims.

### BRIEF DESCRIPTION OF THE DRAWINGS

The present invention will become more fully understood from the detailed description and the accompanying drawings, wherein:

FIG. 1 illustrates a perspective view of a reconstruction plate, in accordance with the general teachings of the present invention;

FIG. 2 illustrates a side elevational view of a reconstruction plate, in accordance with the general teachings of the present invention;

FIG. 3 illustrates a front elevational view of a reconstruction plate, in accordance with the general teachings of the present invention;

FIG. 4 illustrates a bottom plan view of a reconstruction plate, in accordance with the general teachings of the present invention;

FIG. 5 illustrates a partial anterior view of at least a partially reconstructed acetabulum employing the reconstruction plate of the present invention, in accordance with the general teachings of the present invention;

FIG. 6 illustrates a partial basilar view of at least a partially reconstructed acetabulum employing the reconstruction plate of the present invention wherein the femoral head is removed for purposes of illustration, in accordance with the general teachings of the present invention;

FIG. 7 illustrates a partial anterior view of at least a partially reconstructed acetabulum employing the reconstruction plate of the present invention wherein the femoral head is removed for purposes of illustration, in accordance with the general teachings of the present invention;

FIG. 8 illustrates a partial exploded anterior view of at least a partially reconstructed acetabulum employing the reconstruction plate of the present invention wherein the fastening members are shown in detail, in accordance with the general teachings of the present invention;

FIG. 9 illustrates a partial posterior view of at least a partially reconstructed acetabulum employing the reconstruction plate of the present invention wherein portions of the plate are shown in phantom, in accordance with the general teachings of the present invention; and

FIG. 10 illustrates a partial exploded anterior view of at least a partially reconstructed acetabulum employing the reconstruction plate of the present invention wherein portions of the fastening members are shown in detail and the plate is being manipulated to impart a curvature thereto, in accordance with the general teachings of the present invention.

The same reference numerals refer to the same parts throughout the various Figures.

### DETAILED DESCRIPTION OF THE INVENTION

Although the foregoing description is described primarily with respect to the reconstruction of pelvic fractures, especially those involving the acetabulum, and more especially those involving the acetabular dome, it should be appreciated that the present invention is also suitable for the reconstruction of any load-bearing anatomical structure, especially where posterior support of the fractured structure is desirable.

By “anterior aspect,” as that phrase is used herein, it is meant any structure or surface that is not substantially comprised of a posterior aspect of any structure or surface. By way of a non-limiting example, the anterior aspect of an acetabulum or an acetabular dome includes the area extending from the anterior rim of the acetabulum to at least a portion of the iliopubic eminence.

By “posterior aspect,” as that phrase is used herein, it is meant any structure or surface that is not substantially comprised of an anterior aspect of any structure or surface. By way of a non-limiting example, the posterior aspect of an acetabulum or an acetabular dome includes the area extending from at least a portion of the iliopubic eminence to at least the iliopectineal line. By way of another non-limiting example, the posterior aspect of an acetabulum or acetabular dome includes any structure or surface opposite of the acetabulum or acetabular dome.

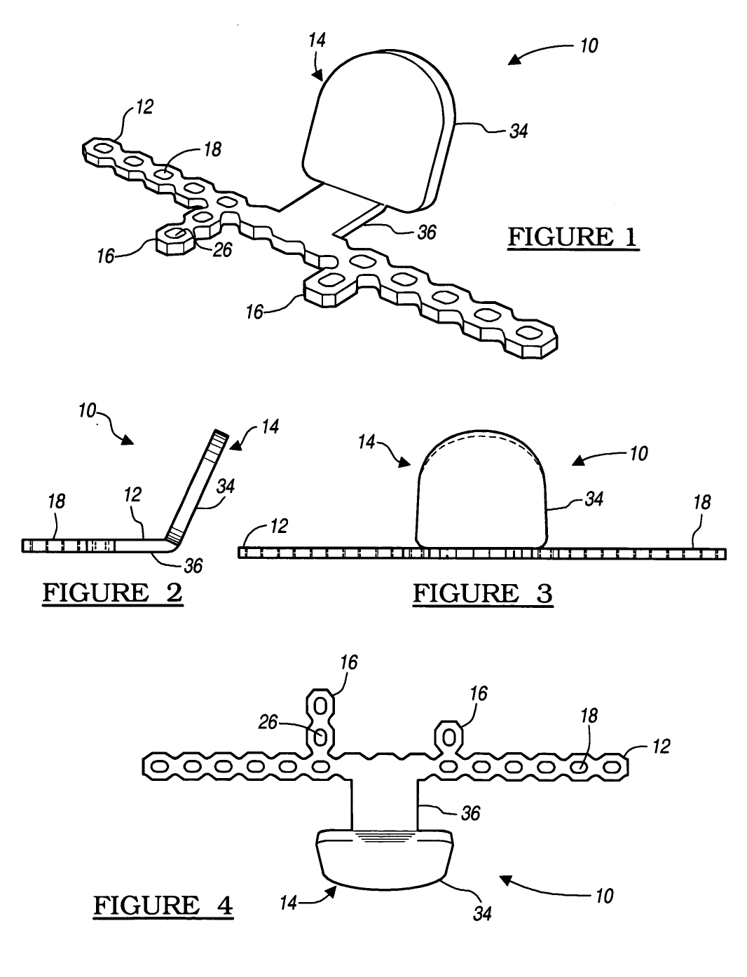

Referring generally to the drawings, and more specifically to FIGS. 1-4, a fixation or reconstruction plate 10 is shown. The reconstruction plate 10 primarily includes a substantially planar attachment member 12 and a support member 14. Optionally, one or more additional attachment members 16 are provided.

The reconstruction plate 10 can be comprised of any number of suitable materials. In accordance with a preferred embodiment of the present invention, the reconstruction plate 10 is preferably comprised of a biocompatible material. In accordance with a more preferred embodiment of the present invention, the reconstruction plate 10 is preferably comprised of a metallic material. In accordance with a highly preferred embodiment of the present invention, the reconstruction plate 10 is preferably comprised of a material selected from the group consisting of stainless steel, titanium, cobalt chrome, and combinations thereof.

In accordance with one embodiment of the present invention, the attachment member 12 and support member 14 are preferably integrally formed to provide a unitary device. In accordance with another embodiment of the present invention, the attachment member 12 and the additional attachment member 16 are preferably integrally formed to provide a unitary device. In accordance with still another embodiment of the present invention, the attachment member 12, support member 14, and additional attachment member 16 are preferably integrally formed to provide a unitary device.

The exact dimensions, e.g., length, height, width, and the like, of the attachment member 12 are not thought to be critical to the success of the present invention, provided that the attachment member 12 is of sufficient dimensions to sufficiently provide a suitable attachment function for the plate 10 without unduly interfering with surrounding tissues (e.g., muscles, tendons, cartilage, arteries, veins, nerves, and the like) around the fracture site to be treated.

By way of a non-limiting example, the attachment member 12 can be constructed in any number of suitable dimensions so as to provide the orthopedic surgeon with a number of different attachment options depending on the particular surgical requirements of the patient.

By way of a non-limiting example, the length of the attachment member 12 is preferably long enough so as to be operable to join or fix two bone fragments together, such as would the case with a fractured acetabulum, but is not so long as to unduly interfere with surrounding tissues (e.g., muscles, tendons, cartilage, arteries, veins, nerves, and the like) around the fracture site to be treated, or alternatively, require the orthopedic surgeon to wastefully cut and remove large portions of the attachment member 12 during surgical procedures, for example.

By way of a non-limiting example, the height of the attachment member 12 should not be so high as to unduly interfere with surrounding tissues (e.g., muscles, tendons, cartilage, arteries, veins, nerves, and the like) around the fracture site to be treated, nor should the height of the attachment member 12 be so low as to potentially compromise the structural strength and durability of the attachment member 12.

By way of a non-limiting example, the width of the attachment member 12 should not be so wide as to unduly interfering with surrounding tissues (e.g., muscles, tendons, cartilage, arteries, veins, nerves, and the like) around the fracture site to be treated, nor should the width of the attachment member 12 be so narrow as to potentially compromise the structural strength and durability of the attachment member 12.

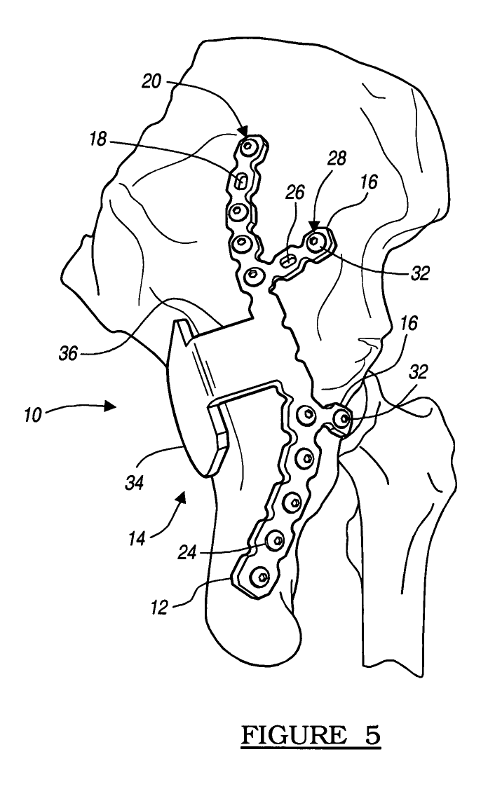

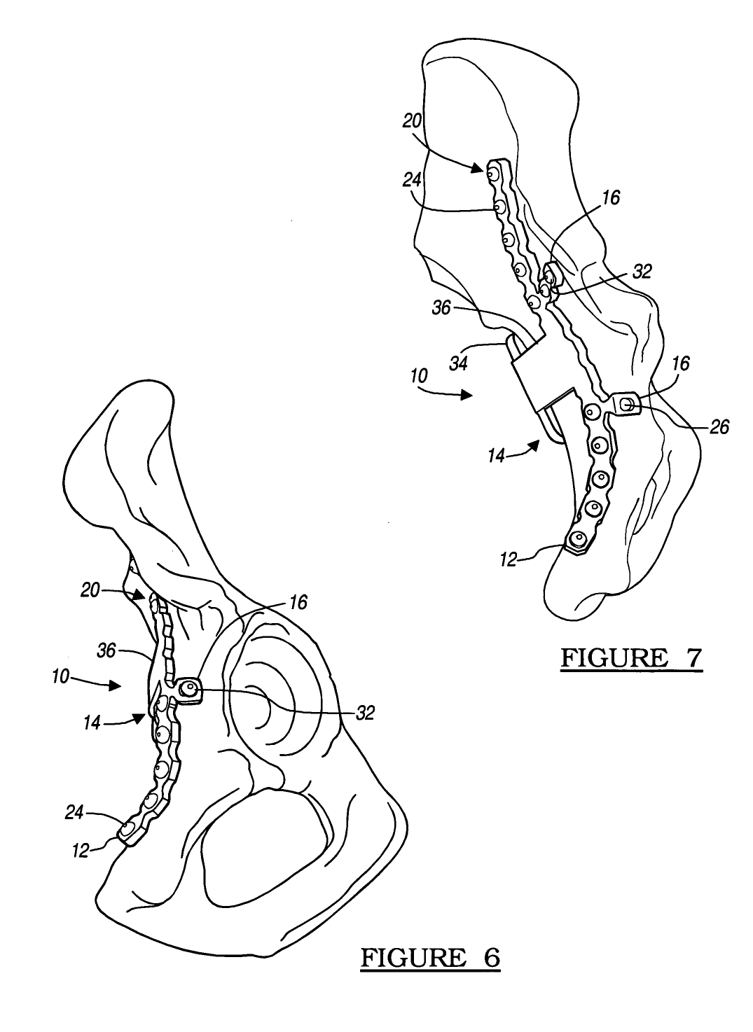

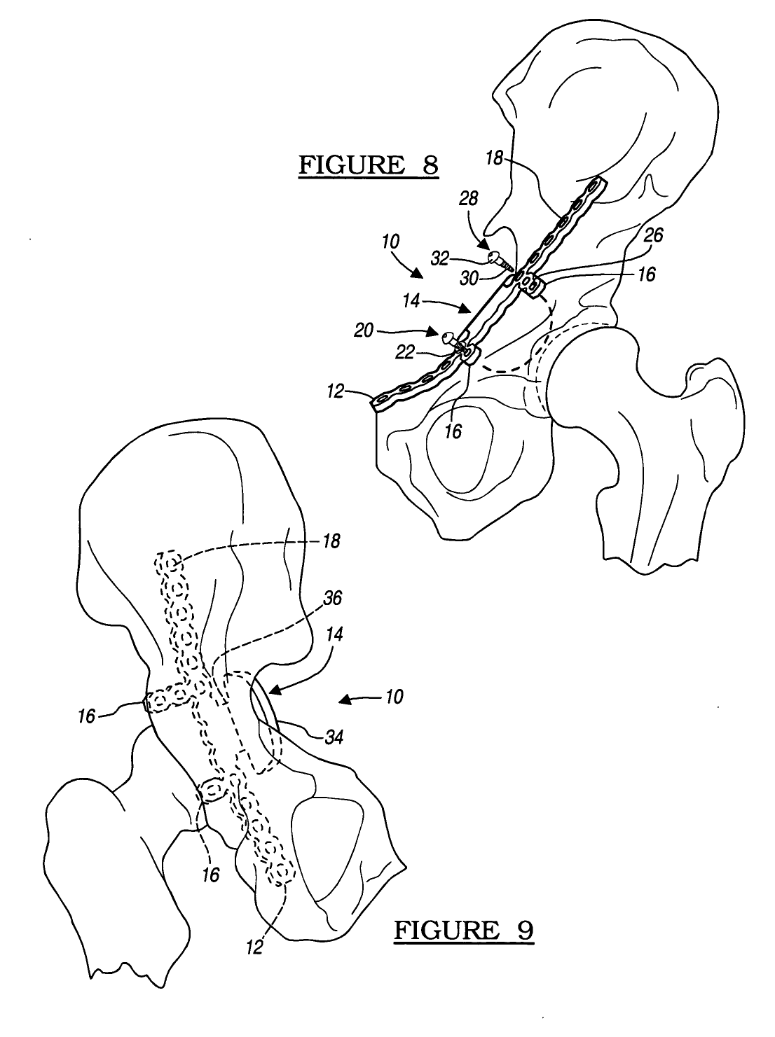

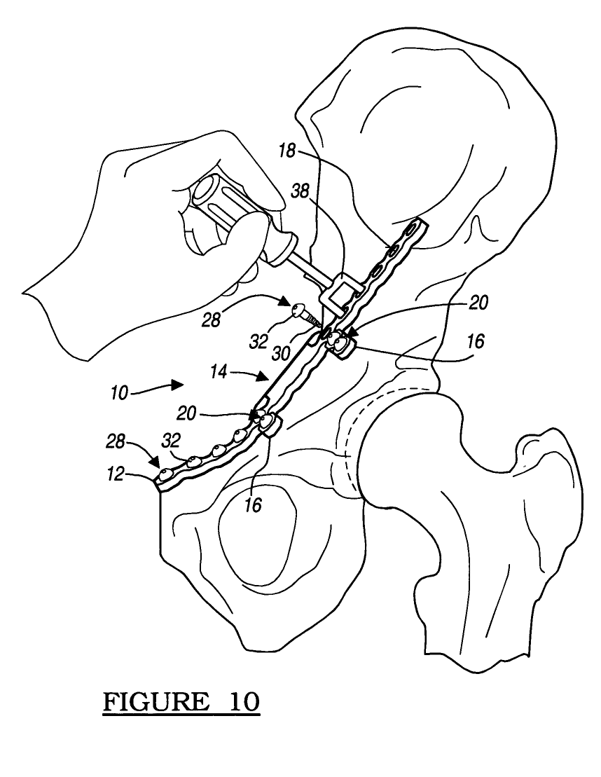

Referring to FIGS. 5-10, the attachment member 12 is preferably provided with at least one, and more preferably a plurality of apertures 18 formed along at least a portion of the length of the attachment member 12. The intended purpose of the aperture 18 is to receive a fastening member 20 (e.g., a bone screw) so as to enable the attachment member 12 to be fastened to an anatomical structure, such as, but not limited to bone, bone fragments, and combinations thereof. Thus, one end of the attachment member 12 may be fastened to a first bone fragment, and a second end of the attachment member 12 may be fastened to a second bone fragment.

Accordingly, the inner diameter of the aperture 18 is preferably wide enough to allow the body 22 of the fastening member 20 to pass therethrough, but not so wide so as to allow the head 24 of the fastening member 20 to pass through as well. The outer diameter of the head 24 of the fastening member 20 is preferably larger than the inner diameter of the aperture 18. In this manner, the head 24 of the fastening member 20 abuts against the surface adjacent to the aperture 18 (e.g., the top surface of the attachment member 12) when the fastening member 20 is substantially fully inserted into the anatomical structure.

Although the attachment member 12 is shown as being substantially straight, it should be appreciated that the attachment member 12 can be manipulated to impart a curvature to the attachment member 12.

The additional attachment member 16 preferably extends away from the attachment member 12. More preferably, the additional attachment member 16 extends angularly (e.g., acutely, obliquely, and the like) away from the attachment member 12. Still more preferably, the additional attachment member 16 substantially perpendicularly away from the attachment member 12. The exact location of the additional attachment member 16 with respect to the attachment member 12 is not thought to be critical to the success of the present invention, provided that the additional attachment member 16 can provide an adequate attachment function for reconstructing the anatomical structure. By way of a non-limiting example, the additional attachment member 16 can be located substantially anywhere along the length of the attachment member 12.

In accordance with a preferred embodiment of the present invention, the additional attachment member 16 is preferably substantially co-planar with respect to the attachment member 12. However, the additional attachment member 16 may be manipulated so as extend away from or towards the attachment member 12 in a non-planar orientation, due, in part, to surgical requirements.

The exact dimensions, e.g., length, height, width, and the like, of the additional attachment member 16 are not thought to be critical to the success of the present invention, provided that the additional attachment member 16 is of sufficient dimensions to sufficiently provide a suitable attachment function for the plate 10 without unduly interfering with surrounding tissues (e.g., muscles, tendons, cartilage, arteries, veins, nerves, and the like) around the fracture site to be treated.

By way of a non-limiting example, the additional attachment member 16 can be constructed in any number of suitable dimensions so as to provide the orthopedic surgeon with a number of different attachment options depending on the particular surgical requirements of the patient.

By way of a non-limiting example, the length of the additional attachment member 16 is preferably long enough so as to be operable to attach to a suitable area of the anatomical structure, relative to attachment member 12, but is not so long as to unduly interfere with surrounding tissues (e.g., muscles, tendons, cartilage, arteries, veins, nerves, and the like) around the fracture site to be treated, or alternatively, require the orthopedic surgeon to wastefully cut and remove large portions of the additional attachment member 16 during surgical procedures, for example.

By way of a non-limiting example, the height of the additional attachment member 16 should not be so high as to unduly interfere with surrounding tissues (e.g., muscles, tendons, cartilage, arteries, veins, nerves, and the like) around the fracture site to be treated, nor should the height of the additional attachment member 16 be so low as to potentially compromise the structural strength and durability of the additional attachment member 16.

By way of a non-limiting example, the width of the additional attachment member 16 should not be so wide as to unduly interfering with surrounding tissues (e.g., muscles, tendons, cartilage, arteries, veins, nerves, and the like) around the fracture site to be treated, nor should the width of the additional attachment member 16 be so narrow as to potentially compromise the structural strength and durability of the additional attachment member 16.

The additional attachment member 16 is preferably provided with at least one, and more preferably a plurality of apertures 26 formed along at least a portion of the length of the additional attachment member 16. The intended purpose of the aperture 26 is to receive a fastening member 28 (e.g., a bone screw) so as to enable the additional attachment member 16 to be fastened to an anatomical structure, such as, but not limited to bone, bone fragments, and combinations thereof.

Accordingly, the inner diameter of the aperture 26 is preferably wide enough to allow the body 30 of the fastening member 28 to pass therethrough, but not so wide so as to allow the head 32 of the fastening member 28 to pass through as well. The outer diameter of the head 32 of the fastening member 28 is preferably larger than the inner diameter of the aperture 26. In this manner, the head 32 of the fastening member 28 abuts against the surface adjacent to the aperture 26 (e.g., the top surface of the additional attachment member 16) when the fastening member 28 is substantially fully installed into the anatomical structure.

Although the additional attachment member 16 is shown as being substantially straight, it should be appreciated that the additional attachment member 16 can be manipulated to impart a curvature to the additional attachment member 16.

The support member 14 is intended to preferably support an anatomical structure, more preferably support a posterior aspect of an anatomical structure, still more preferably support a posterior aspect of an acetabulum, and most preferably a posterior aspect of an acetabular dome.

The support member 14 preferably includes a paddle member 34 angularly extending from the attachment member 12. Although a substantially paddle-shaped member is shown, it should be appreciated that other configurations may be employed with respect to the paddle member 34.

An optional extension member 36 may be provided between the paddle member 34 and the attachment member 12. The intended purpose of the extension member 36 is to allow the paddle member 34 to be extended a sufficient distance from the attachment member 12 so as to be properly positioned with respect to the anatomical structure to be supported.

In accordance with a preferred embodiment of the present invention, the extension member 36 extends substantially perpendicularly from the attachment member 12. In accordance with a preferred embodiment of the present invention, the extension member 36 is substantially coplanar with respect to the attachment member 12, whereas the paddle member 34 is not substantially coplanar with respect to the attachment member 12.

In accordance with a preferred embodiment of the present invention, the paddle member 34 extends substantially perpendicularly from the extension member 36 so as to form an angle therebetween. The angle formed between the extension member 36 and the paddle member 34 will be dependent, in part, on the anatomical structure to be supported.

The exact location of the support member 14 with respect to the attachment member 12 is not thought to be critical to the success of the present invention, provided that the support member 14 can provide adequate support to the anatomical structure, and particularly to the posterior aspect of the anatomical structure, to be supported. In accordance with a preferred embodiment of the present invention, the support member 14 is located substantially at or near the midpoint of the attachment member 12.

The support member 14, and especially the paddle member 34 is preferably located adjacent to, and more preferably in abutting engagement with, the anatomical structure, and particularly to the posterior aspect of the anatomical structure, to be supported. Thus, when a load is applied to the anatomical structure, and particularly to the posterior aspect of the anatomical structure, to be supported, the support member 14, and especially the paddle member 34, provides support, e.g., mechanical support, to the anatomical structure, and particularly to the posterior aspect of the anatomical structure, to be supported. In this manner, an anatomical structure reconstructed with the reconstruction plate 10 of the present invention will be provided with enhanced posterior mechanical support so as to prevent re-fractures of the anatomical structure, especially during loading.

By way of a non-limiting example, the reconstruction plate 10 is particularly suitable for reconstructing fractures of the acetabulum, especially those involving the acetabular dome (e.g., central acetabulum fractures). In particular, the reconstruction plate 10 of the present invention is intended to replace conventional anterior pelvic reconstruction plates. In most circumstances, it will also be necessary to employ a conventional posterior pelvic reconstruction plate in conjunction with the reconstruction plate 10 of the present invention when reconstructing acetabular fractures.

By way of a non-limiting example, the support member 14, and more particularly the paddle member 34, is located in proximity to, and preferably in abutting engagement with, the posterior aspect of the acetabulum, and more particularly the posterior aspect of the acetabular dome. By way of a non-limiting example, the posterior aspect of the acetabulum and/or acetabular dome includes the area extending from at least a portion of the iliopubic eminence to at least the iliopectineal line. By way of another non-limiting example, the posterior aspect of the acetabulum and/or acetabular dome includes any structure or surface opposite of the acetabulum or acetabular dome.

In this manner, when the reconstructed acetabular dome is loaded, e.g., by the femoral head, during standing, walking, running, jumping, and the like, the reconstructed acetabular dome will be provided with adequate mechanical support so as to at least lessen the likelihood of re-fracture of the acetabular dome and potential subsequent penetration of the femoral head into the pelvis.

Once the support member 14 is properly located, one or more fastening members 20 are inserted through the apertures 18 of the attachment member 12 and fastened to the underlying bone tissue. Preferably, the attachment member 12 is fastened to the anterior aspect of the anatomical structure. More preferably, the attachment member 12 is fastened to the anterior aspect of an acetabulum. Still more preferably, the attachment member 12 is fastened to the anterior aspect of an acetabular dome. By way of a non-limiting example, the anterior aspect includes the area extending from the anterior rim of the acetabulum to at least a portion of the iliopubic eminence.

As previously described, the attachment member 12 may be manipulated preferably after at least one fastening member 20 has been installed, to conform to a desired configuration, depending, in part, on the particular surgical requirements of the patient. Previously, concurrently or subsequently to this, one or more fastening members 28 are inserted through the apertures 26 of the additional attachment member 16 and fastened to the underlying bone tissue. Preferably, the additional attachment member 16 is fastened to the anterior aspect of the anatomical structure. More preferably, the additional attachment member 16 is fastened to the anterior aspect of an acetabulum. Still more preferably, the additional attachment member 16 is fastened to the anterior aspect of an acetabular dome. By way of a non-limiting example, the anterior aspect includes the area extending from the anterior rim of the acetabulum to at least a portion of the iliopubic eminence.

As previously described, the additional attachment member 16 may be manipulated, preferably after at least one fastening member 28 has been installed, to conform to a desired configuration, depending, in part, on the particular surgical requirements of the patient. The manipulation can be accomplished by any number of conventional reconstruction plate tools 38 by selectively applying force from any number of directions so as to curve the respective attachment members in several different directions, if desired.

In this manner, the present invention provides a device and method for reconstructing a fracture involving the acetabulum, especially the acetabular dome, so as to provide adequate support to a posterior aspect of the reconstructed acetabulum, especially the reconstructed acetabular dome.

The foregoing description is considered illustrative only of the principles of the invention. Furthermore, because numerous modifications and changes will readily occur to those skilled in the art, it is not desired to limit the invention to the exact construction and process shown as described above. Accordingly, all suitable modifications and equivalents that may be resorted to that fall within the scope of the invention as defined by the claims that follow.

1. A reconstruction plate for reconstructing a fractured load-bearing anatomical structure, comprising:
an attachment member including at least one area defining an aperture formed therein; and
a support member extending angularly from the attachment member;
wherein the support member is operable to provide support to a posterior aspect of the reconstructed anatomical structure during loading of the reconstructed anatomical structure.

2. The invention according to claim 1, wherein the anatomical structure is an acetabulum.

3. The invention according to claim 1, wherein the anatomical structure is an acetabular dome.

4. The invention according to claim 1, wherein the attachment member includes a plurality of areas defining apertures formed therein.

5. The invention according to claim 1, wherein the reconstruction plate is comprised of a biocompatible material.

6. The invention according to claim 1, wherein the reconstruction plate is comprised of a metallic material.

7. The invention according to claim 1, wherein the reconstruction plate is comprised of materials selected from the group consisting of stainless steel, titanium, cobalt chrome, and combinations thereof.

8. The invention according to claim 1, wherein the attachment member is operable to be manipulated so as to impart a curvature to at least a portion of the attachment member.

9. The invention according to claim 1, wherein the attachment member and the support member are integrally formed.

10. The invention according to claim 1, wherein the aperture is operable to receive a fastening member.

11. The invention according to claim 1, wherein the aperture is operable to receive a bone screw.

12. The invention according to claim 1, wherein the attachment member includes a first end and a second end, the first end being operable to be fastened to a first bony member and the second end being operable to be fastened to a second bony member.

13. The invention according to claim 1, wherein the support member is in proximity to a posterior aspect of the anatomical structure.

14. The invention according to claim 1, wherein the support member is in proximity to a posterior aspect of an acetabulum.

15. The invention according to claim 1, wherein the support member is in proximity to a posterior aspect of an acetabular dome.

16. The invention according to claim 1, wherein the attachment member is operable to be fastened to an anterior aspect of the anatomical structure.

17. The invention according to claim 1, further comprising at least one other attachment member extending from the attachment member, the at least one other attachment member including at least one area defining an aperture formed therein.

18. The invention according to claim 17, wherein the at least one other attachment member extends substantially perpendicularly from the attachment member.

19. The invention according to claim 17, wherein the at least one other attachment member is substantially coplanar to the attachment member.

20. The invention according to claim 17, wherein the at least one other attachment member includes a plurality of areas defining apertures formed therein.

21. The invention according to claim 17, wherein the attachment member and the at least one other attachment member are integrally formed.

22. The invention according to claim 17, wherein the aperture is operable to receive a fastening member.

23. The invention according to claim 17, wherein the aperture is operable to receive a bone screw.

24. The invention according to claim 17, wherein the at least one other attachment member is operable to be fastened to an anterior aspect of the anatomical structure.

25. A reconstruction plate for reconstructing a fractured acetabulum, comprising:
an attachment member including at least one area defining an aperture formed therein; and
a support member extending angularly from the attachment member;
wherein the support member is operable to provide support to a posterior aspect of the reconstructed acetabulum during loading of the reconstructed acetabulum.

26. The invention according to claim 25, wherein the attachment member includes a plurality of areas defining apertures formed therein.

27. The invention according to claim 25, wherein the reconstruction plate is comprised of a biocompatible material.

28. The invention according to claim 25, wherein the reconstruction plate is comprised of a metallic material.

29. The invention according to claim 25, wherein the reconstruction plate is comprised of materials selected from the group consisting of stainless steel, titanium, cobalt chrome, and combinations thereof.

30. The invention according to claim 25, wherein the attachment member is operable to be manipulated so as to impart a curvature to at least a portion of the attachment member.

31. The invention according to claim 25, wherein the attachment member and the support member are integrally formed.

32. The invention according to claim 25, wherein the aperture is operable to receive a fastening member.

33. The invention according to claim 25, wherein the aperture is operable to receive a bone screw.

34. The invention according to claim 25, wherein the attachment member includes a first end and a second end, the first end being operable to be fastened to a first bony member and the second end being operable to be fastened to a second bony member.

35. The invention according to claim 25, wherein the support member is in proximity to a posterior aspect of an acetabular dome.

36. The invention according to claim 25, wherein the attachment member is operable to be fastened to an anterior aspect of the acetabulum.

37. The invention according to claim 25, further comprising at least one other attachment member extending from the attachment member, the at least one other attachment member including at least one area defining an aperture formed therein.

38. The invention according to claim 37, wherein the at least one other attachment member extends substantially perpendicularly from the attachment member.

39. The invention according to claim 37, wherein the at least one other attachment member is substantially coplanar to the attachment member.

40. The invention according to claim 37, wherein the at least one other attachment member includes a plurality of areas defining apertures formed therein.

41. The invention according to claim 37, wherein the attachment member and the at least one other attachment member are integrally formed.

42. The invention according to claim 37, wherein the aperture is operable to receive a fastening member.

43. The invention according to claim 37, wherein the aperture is operable to receive a bone screw.

44. The invention according to claim 37, wherein the at least one other attachment member is operable to be fastened to an anterior aspect of the acetabulum.

45. A reconstruction plate for reconstructing a fractured acetabulum, comprising:
an attachment member including a plurality of areas defining apertures formed therein;
at least one other attachment member extending substantially perpendicularly from the attachment member, the at least one other attachment member including at least one area defining an aperture formed therein; and
a support member extending angularly from the attachment member;
wherein the support member is operable to provide support to a posterior aspect of the reconstructed acetabulum during loading of the reconstructed acetabulum.

46. The invention according to claim 45, wherein the reconstruction plate is comprised of a biocompatible material.

47. The invention according to claim 45, wherein the reconstruction plate is comprised of a metallic material.

48. The invention according to claim 45, wherein the reconstruction plate is comprised of materials selected from the group consisting of stainless steel, titanium, cobalt chrome, and combinations thereof.

49. The invention according to claim 45, wherein the attachment member is operable to be manipulated so as to impart a curvature to at least a portion of the attachment member.

50. The invention according to claim 45, wherein the attachment member, the at least one other attachment member, and the support member are integrally formed.

51. The invention according to claim 45, wherein any aperture is operable to receive a fastening member.

52. The invention according to claim 45, wherein any aperture is operable to receive a bone screw.

53. The invention according to claim 45, wherein the attachment member includes a first end and a second end, the first end being operable to be fastened to a first bony member and the second end being operable to be fastened to a second bony member.

54. The invention according to claim 45, wherein the support member is in proximity to a posterior aspect of an acetabular dome.

55. The invention according to claim 45, wherein the at least one other attachment member is substantially coplanar to the attachment member.

56. The invention according to claim 45, wherein the at least one other attachment member includes a plurality of areas defining apertures formed therein.

57. The invention according to claim 45, wherein the attachment member is operable to be fastened to an anterior aspect of the acetabulum.

58. The invention according to claim 45, wherein the at least one other attachment member is operable to be fastened to an anterior aspect of the acetabulum.

59. A method for reconstructing a fractured load-bearing anatomical structure, comprising:
providing a reconstruction plate, comprising:
an attachment member including at least one area defining an aperture formed therein; and
a support member extending angularly from the attachment member; and
fastening the reconstruction plate in proximity to the anterior aspect of the fractured anatomical structure so as to at least partially reconstruct the fractured anatomical structure;
wherein the support member is operable to provide support to the posterior aspect of the reconstructed anatomical structure during loading of the reconstructed anatomical structure.

60. The invention according to claim 59, wherein the reconstruction plate is comprised of a biocompatible material.

61. The invention according to claim 59, wherein the reconstruction plate is comprised of a metallic material.

62. The invention according to claim 59, wherein the reconstruction plate is comprised of materials selected from the group consisting of stainless steel, titanium, cobalt chrome, and combinations thereof.

63. The invention according to claim 59, further comprising manipulating the attachment member so as to impart a curvature to at least a portion of the attachment member.

64. The invention according to claim 59, wherein the support member is in proximity to a posterior aspect of an acetabulum.

65. The invention according to claim 59, wherein the support member is in proximity to a posterior aspect of an acetabular dome.

66. The invention according to claim 59, wherein the attachment member is fastened to an anterior aspect of the anatomical structure.

67. The invention according to claim 59, further comprising providing at least one other attachment member extending from the attachment member, the at least one other attachment member including at least one area defining an aperture formed therein.

68. The invention according to claim 67, wherein the at least one other attachment member extends substantially perpendicularly from the attachment member.

69. The invention according to claim 67, wherein the at least one other attachment member is substantially coplanar to the attachment member.

70. The invention according to claim 67, wherein the at least one other attachment member includes a plurality of areas defining apertures formed therein.

71. The invention according to claim 67, wherein the at least one other attachment member is fastened to an anterior aspect of the anatomical structure.

72. A method for reconstructing a fractured acetabulum, comprising:
providing a reconstruction plate, comprising:
an attachment member including at least one area defining an aperture formed therein; and
a support member extending angularly from the attachment member; and
fastening the reconstruction plate in proximity to the anterior aspect of the fractured acetabulum so as to at least partially reconstruct the fractured acetabulum;
wherein the support member is operable to provide support to the posterior aspect of the reconstructed acetabulum during loading of the reconstructed acetabulum.

73. The invention according to claim 72, wherein the reconstruction plate is comprised of a biocompatible material.

74. The invention according to claim 72, wherein the reconstruction plate is comprised of a metallic material.

75. The invention according to claim 72, wherein the reconstruction plate is comprised of materials selected from the group consisting of stainless steel, titanium, cobalt chrome, and combinations thereof.

76. The invention according to claim 72, further comprising manipulating the attachment member so as to impart a curvature to at least a portion of the attachment member.

77. The invention according to claim 72, wherein the support member is in proximity to a posterior aspect of an acetabulum.

78. The invention according to claim 72, wherein the support member is in proximity to a posterior aspect of an acetabular dome.

79. The invention according to claim 72, wherein the attachment member is operable to be fastened to an anterior aspect of the acetabulum.

80. The invention according to claim 72, further comprising providing at least one other attachment member extending from the attachment member, the at least one other attachment member including at least one area defining an aperture formed therein.

81. The invention according to claim 80, wherein the at least one other attachment member extends substantially perpendicularly from the attachment member.

82. The invention according to claim 80, wherein the at least one other attachment member is substantially coplanar to the attachment member.

83. The invention according to claim 80, wherein the at least one other attachment member includes a plurality of areas defining apertures formed therein.

84. The invention according to claim 80, wherein the at least one other attachment member is operable to be fastened to an anterior aspect of the acetabulum.
